(19) United States
(12) Patent Application Publication
(10) Pub. No.: US 2010/0288270 A1
(43) Pub. Date: Nov. 18, 2010

(54) LIQUID EJECTING HEAD

(75) Inventors: Hiroyuki Wada, Machida-shi (JP); Yoshihisa Negishi, Osato-gun (JP)

Correspondence Address: FITZPATRICK CELLA HARPER & SCINTO, 1290 Avenue of the Americas, NEW YORK, NY, 10104-3800, US

(73) Assignee: CANON KABUSHIKI KAISHA, Tokyo (JP)

(21) Appl. No.: 12/376,056

(22) PCT Filed: Dec. 5, 2008
(86) PCT No.: PCT/JP2008/072584

§ 371 Date: Feb. 2, 2009

(30) Foreign Application Priority Data
Dec. 7, 2007 (JP) 2007-317085

(51) Int. Cl. A61M 11/00

(52) U.S. Cl. 128/200.14

(57) ABSTRACT

A liquid ejecting head includes an energy generating element for generating energy utilized for ejecting liquid containing a medicine, and a plurality of ejection outlets, provided for said energy generating element, for ejecting the liquid. Each of the ejection outlets has a rotationally asymmetrical cross-section in a plane parallel to the energy generating element.

### TECHNICAL FIELD

The present invention relates to a liquid ejecting head for ejecting liquid in the form of a minute liquid droplet and particularly relates to a liquid ejecting head suitably used for an inhaling device used for atomizing a solution containing a medicine so that the medicine is inhaled into lungs in the field of medicine.

### BACKGROUND ART

A liquid ejecting head for ejecting liquid in the form of a minute liquid droplet has been conventionally used widely as an ink jet (recording) head in the field of ink jet recording. The ink jet head is required to not only simply eject liquid droplets but also ensure stability of a liquid droplet ejection direction. With respect to the conventional ink jet head, various proposals have been made to meet these requirements.

For example, U.S. Pat. No. 5,754,202 discloses an ink jet head in which a recessed portion is formed at a periphery of ejection outlets for ejecting ink to prevent contact of ink between adjacent ejection outlets and thereby to ensure stable ink flying.

Further, Japanese Laid-Open Patent Application (JP-A) Hei 5-193141 and JP-A Hei 11-334069 disclose ink jet heads in which an ejection outlet forming member having a recessed portion wherein ejection outlets are formed and an inner surface of the recessed portion is subjected to ink affinity treatment and in which a surface of the ejection outlet forming member excluding the recessed portion is subjected to ink-repellent treatment and ink is ejected in a state in which a meniscus is formed at the recessed portion to permit stable ejection of liquid droplets and improved recording quality.

Further, U.S. Pat. No. 6,926,392 discloses that it is possible to eject a plurality of minute liquid droplets at the same time by providing plurality of ejection outlets with respect to one energy generating element. In U.S. Pat. No. 6,926,392, as an application of the liquid ejecting head to a device other than a recording head, the use of the liquid ejecting head in an inhaling device of a solution containing a medicine has also been studied. With respect to liquid ejected from the inhaling device, a reaching position in a respiratory system is roughly determined by a liquid droplet diameter of the liquid. For example, in order to diameter of 6-9 μm is effective. Further, it has been known that the liquid is most liable to reach a throat when the liquid droplet diameter is 5-6 μm, to reach a bronchus when the liquid droplet diameter is 3-5 μm, and to reach an alveolus when the liquid droplet diameter is 3 μm or less. Therefore, an optimum liquid droplet diameter varies depending on a target location, so that the inhaling device is required to have a performance such that the liquid droplets are uniformly and stably ejected. In this regard, the liquid ejecting head is capable of ejecting the liquid droplets easily and uniformly by, adjusting the ejection outlets or the like.

Further, the liquid ejecting, head used in the inhaling device used during inhalation of the liquid medicine in the atomized form into the lungs is required to not only exhibit the above-described uniform liquid droplet forming performance but also realize a high density of the ejection outlets in order to increase the number of ejected liquid droplets.

However, the conventional methods have been accompanied with such a problem that the ejected liquid droplets contact each other to increase the liquid droplet diameter by decreasing the distance between adjacent ejection outlets for ejecting the liquid droplets, so that intended minute liquid droplets cannot be obtained. For example, in the case where the plurality of ejection outlets is provided to one energy generating element as described in U.S. Pat. No. 6,926,392, the distance between adjacent ejection outlets is decreased. As a result, it can be assumed that ejected liquid droplets contact each other.

### DISCLOSURE OF THE INVENTION

In view of the above-described problem, the present invention has been accomplished. A principal object of the present invention is to provide a liquid ejecting head which does not cause mutual contact of ejected liquid droplets even in the case where a plurality of ejection outlets is provided with respect to one energy generating element.

According to an aspect of the present invention, there is provided a liquid ejecting head comprising:

an energy generating element for generating energy for ejecting liquid containing a medicine; and

a plurality of ejection outlets, provided for said energy generating element, for ejecting the liquid,

wherein each of the ejection outlets has a rotationally asymmetrical cross-section in a plane parallel to the energy generating element.

These and other objects, features and advantages of the present invention will become more apparent upon a consideration of the following description of the preferred embodiments of the present invention taken in conjunction with the accompanying drawings.

### BRIEF DESCRIPTION OF THE DRAWINGS

FIG. 1 is a perspective view of an outer to appearance of a liquid ejecting head device according to an embodiment of the present invention.

FIG. 2 is an exploded perspective view of the liquid ejecting head device shown in FIG. 1.

FIG. 3 is a partly broken perspective view of a heat generating element substrate shown in FIG. 2.

FIG. 4A is a plan perspective view showing a liquid ejecting head according to the present invention, FIG. 4B is a sectional view taken along X-X′ line shown in FIG. 4A, and FIG. 4C is an enlarged plan view of an ejection outlet shown in FIG. 4A.

FIGS. 5A to 5E are schematic views for illustrating a state of liquid ejection viewed from the X-X′ cross section of the liquid ejecting head shown in FIG. 4B.

FIG. 6A is a plan perspective view showing a modified embodiment of the liquid ejecting head shown in FIGS. 4A to 4C, and FIG. 6B is a sectional view taken along X-X′ line shown in FIG. 6A.

FIGS. 7A to 7E are schematic views for illustrating a state of liquid ejection viewed from the X-X′ cross section of the liquid ejecting head shown in FIG. 6B.

FIG. 8A is a plan perspective view showing a modified embodiment of ejection outlets shown in FIGS. 6A and 6B, and FIG. 8B is a sectional view taken along X-X′ line shown in FIG. 8A.

FIGS. 9A and 9B show a part of an ejection outlet group shown in FIG. 3, wherein FIG. 9A shows a modified embodiment of the ejection outlets shown in FIGS. 4A to 4C and an arrangement view and

FIG. 9B shows a modified embodiment of the ejection outlets shown in FIGS. 6A and 6B and an arrangement view.

### BEST MODE FOR CARRYING OUT THE INVENTION

The liquid ejecting head of the present invention can be suitably used for, a device for ejecting (or spraying) minute liquid droplets. Specific applications thereof are not particularly limited but may include an inhaling device used for dosing a respiratory system of a patient with a solution containing a medicine in the field of medicine. Other applications may include a steamer, spray of paint or the like, etc.

In the case where the liquid ejecting head of the present invention is used as a head for a medicine inhaling device, it is possible to employ a constitution in which a device portion at which the liquid ejecting head is disposed is connected to a dispenser for the medicine.

The liquid (solution) described above may be in a liquid form and is not particularly limited but may, e.g., preferably be a medicine-containing solution, particularly a specially formulated solution for being administered to the respiratory system of the patient. The liquid form may include a state such as sol or gel.

The medicine described above refers to a substance which exerts some action or effect on animals such as mammals and is not particularly limited. For example, the medicine may include: proteins such as insulin, human growth hormone and gonadotrophic hormone: nicotine; antiasthmatic; and anesthetic.

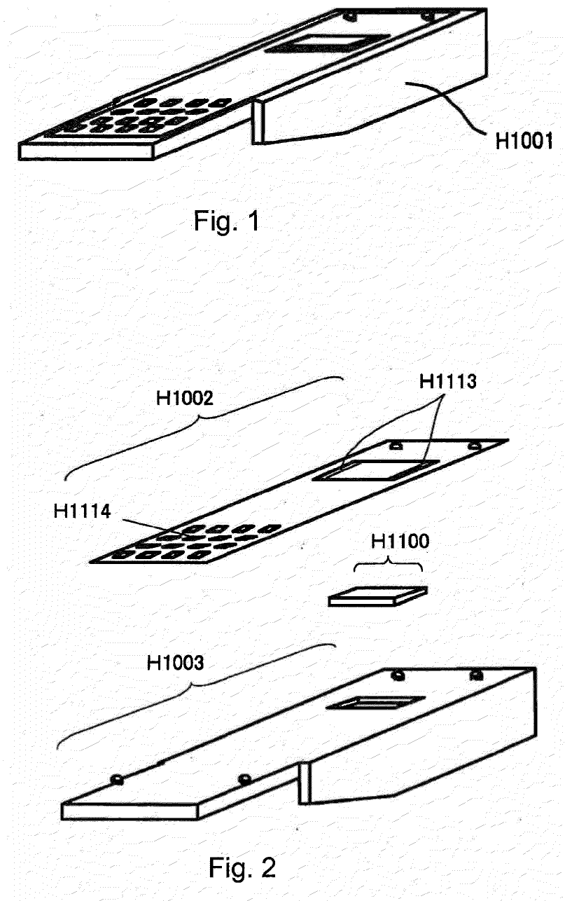

First, a constitution of the liquid ejecting head device will be described with reference to FIGS. 1 to 3. FIGS. 1 to 3 are schematic views for illustrating the liquid ejecting head device using heat generating elements suitable for carrying out the present invention.

### (Liquid Ejecting Head Device)

FIG. 1 shows a conventional liquid ejecting head device H1001 but in the present invention, this device constitution can be utilized as, e.g., an inhaling device. The liquid ejecting head device H1001 is a bubble eject device of a side shooter type in which ejection is performed by using a heat generating element for generating thermal energy for causing film boiling of liquid depending on an electric signal. In the following description, the heat generating element is used but particularly an energy generating element is not limited to the heat generating element and it is also possible to use a vibration energy generating element such as a piezoelectric element.

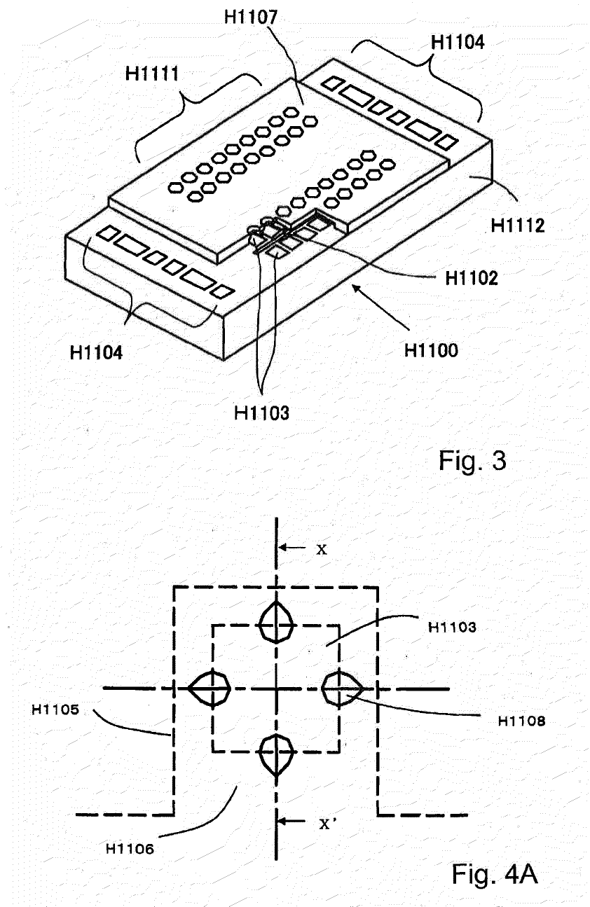

Next, respective constituents constituting the liquid ejecting head device H1001 will be described in order. FIG. 2 is an exploded perspective view of the liquid ejecting head device of FIG. 1. The liquid ejecting head device H1001 is, as shown in FIG. 2, constituted by a heat generating element substrate H1100, an electric wiring tape 1002 and a container holder H1003.

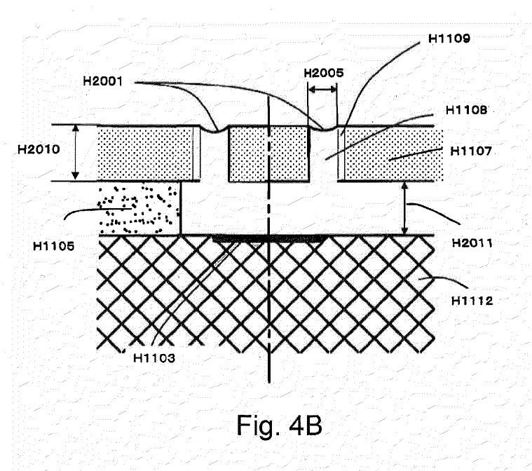

First, the container holder H1003 will be described. The container holder H1003 is, e.g., formed by resin molding. The container holder H1003 includes a connecting port for introducing liquid from a detachable liquid container (not shown) to a liquid supply port (FIG. 3) and also partly has a function of retaining the detachable liquid container (not shown). In this container, e.g., a solution containing a medicine is contained and stored.

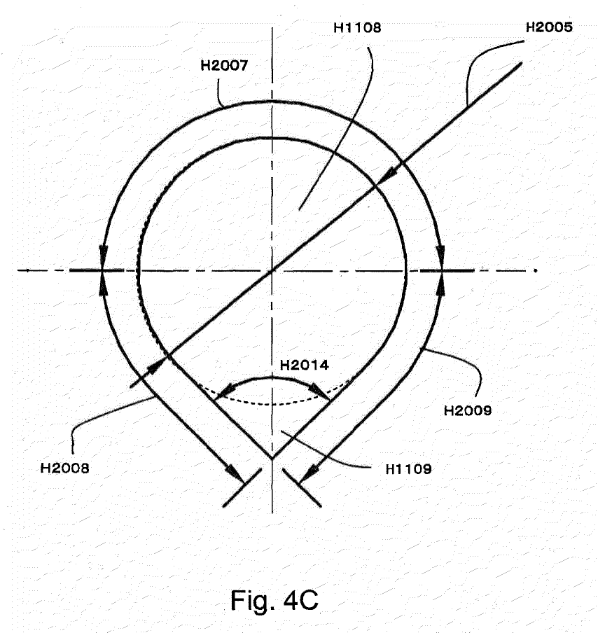

FIG. 3 is a partly broken perspective view for illustrating a constitution of the heat generating element substrate H1100. The heat generating element substrate H1100 is prepared by forming the liquid supply port H1002 consisting of an elongated groove-like through hole as a liquid flow passage on, e.g., an Si substrate in a thickness of 0.5-1 mm by anisotropic etching utilizing crystal orientation or by sand-blast. Further, on each of both sides of the liquid supply port H1102, an array of heat generating elements H1103 is arranged and unshown electric wiring for supplying electric power to the heat generating element is formed of Au or the like through a film-forming technique. Further, each at both end portions of the heat generating element substrate 1100, an electrode portion H1104 for supplying electric power to the electric wiring is disposed. On the Si substrate H1112, a liquid flow passage wall H1105 (FIGS. 4A and 4B) and ejection outlets H1108 (FIGS. 4A and 4B) which are configured to form a liquid flow passage correspondingly to the heat generating element H1103 are formed by a lithographic technique with a resin material and a micro fabrication technique, thus forming a ejection outlet group H1111.

The liquid supplied from the liquid supply port H1102 is ejected from the ejection outlets H1108, provided oppositely to the heat generating element H1103, by bubbles generated by the heat generating element H1103.

The heat generating element substrate H1100 is bonded to the container holder H1003 by a first adhesive. The first adhesive may desirably have a low curing temperature, a short curing time, a relatively high hardness after curing, and a liquid-resistant property. For example, a normal temperature curable adhesive and UV curable adhesive which contain a silicone as a main component can be used.

Next, the electric wiring tape G1002 will be described. The electric wiring tape H1002 applies an electric signal, for ejecting liquid, to the heat generating element substrate H1100. The electric wiring tape H1002 includes an opening for incorporating the heat generating element substrate H1100, electrode terminal H1113 corresponding to the electrode portions H1104 of the heat generating element substrate H1100, and external signal input terminals H1114 which is located, at an end, portion of the electric wiring tape H1002 and is configured to receive an electric signal from a main assembly of an apparatus. The external signal input terminals H1114 are connected to the electrode terminals H1113 by a continuous wiring pattern of a copper foil or the like (not shown). The electric wiring tape H1002 is bonded to the container holder H1003 with a second adhesive at a back surface of the electric wiring tape H1002. The second adhesive may desirably have a low curing temperature and a short curing time. For example, hot-melt adhesive may be used.

The electric wiring tape 1002 and the heat generating element substrate H1100 are electrically connected to each other. A connecting method may be electric connection by thermo-ultrasonic pressure bonding between the electrode portions H1004 of the heat generating element substrate H1100 and the electrode terminals H1113 of the electric wiring tape H1002 by using, e.g., an Au wire having a diameter of 50 μm or may be electric connection by thermo-ultrasonic pressure bonding similarly by using the Au wire having a diameter of 50 μm.

The wire by which the electrode terminals H1113 of the electric wiring tape H1002 and the electrode portions H 1104 of the heat generating element substrate H1100 are electrically connected is coated with a third adhesive. As the third adhesive, e.g., a normal temperature curable adhesive and UV curable adhesive which contain a silicone as a main component can be used. Thus, the liquid ejecting head device H1001 is completed by assembling the heat generating element substrate H1100 and the electric wiring tape H1002 on the container holder H1003.

Hereinafter, the present invention will be described specifically based on several Embodiments,

### Embodiment 1

### Liquid Ejecting Head

FIGS. 4A to 4C show an example of a liquid ejecting head in this embodiment, wherein FIG. 4A is a plan perspective view of the liquid ejecting head as seen in a direction perpendicular to the substrate of the liquid ejecting head, FIG. 4B is a sectional view taken along X-X′ line shown in FIG. 4A, and FIG. 4C is an enlarged view of an ejection outlet shown in FIG. 4A.

In the liquid ejecting head shown in FIGS. 4A to 4C, a heat generating element H1103 as an energy generating element for ejecting liquid is provided on an Si substrate H1112. The heat generating element H1103 is disposed correspondingly to a liquid flow passage H1106. In FIG. 4A, only a single heat generating element H1103 and a single liquid flow passage H1106 are shown but actually, a plurality of heat generating elements H1103 is disposed on one Si substrate H1112 and a plurality of liquid flow passages H1106 is disposed correspondingly to associated ones of the heat generating elements H1103. That is, on the substrate, a plurality of liquid ejecting heads is disposed. The energy generating element is not limited to the heat generating element such as a heater but may also be a vibration energy generating element such as a heater but may also be a vibration energy generating element such as a piezoelectric elements. Further, in place of the Si substrate H1112, e.g., the substrate can also be formed of glass, ceramics, a resin material, metals other than Si, and the like.

The liquid flow passage 1106 is defined by surrounding members including an ejection outlet plate H1107 provided with a plurality of ejection outlets H1108 for ejecting liquid in the form of liquid droplets, the Si substrate H1112, and a liquid flow passage wall H1105 defining a space between the ejection outlet plate H1107 and the Si substrate H1112.

FIG. 4C is the enlarged vie of each ejection outlet. A cross-sectional configuration of a plane, of the ejection outlet H1108, which is parallel to a surface of the heat generating element (hereinafter referred to as an “ejection outlet cross-sectional configuration”) has no rotation symmetry and includes a cut-away portion H1109. The shape of the ejection outlet is obtained by boring or hollowing out the ejection outlet plate in accordance with the ejection outlet cross-sectional configuration with respect to a direction perpendicular to the heat generating element surface.

Here, specific dimensions of the liquid ejecting head will be described but the present invention is not limited thereto.

The heat generating element H1103 is a square having each side of 10 μm. The ejection outlets H1108 are disposed on the respective sides of the square heat generating element H1003 at positions corresponding to outermost effective ejection power positions so that a center of an opening diameter H2005 (FIG. 4C) of each ejection outlet 1108 is located on each side of the square heat generating element H1003.

The opening diameter H2005 of the ejection outlet H1108 is 3 μm and a semicircular circumferential length H2007 is about 4.7 μm (diameter H2005)×π/2). Each of lengths H2008 and H2009 from a deepest point (or a vertex (apex)) of the cut-away portion H1109 to an end point of the semicircular circumferential length H2007 is about 2.7 μm ((diameter H2005)×π/8+1.5), Further, a vertex (apex) angle H2014 of the cut-away portion H1109 is 90 degrees. The ejection outlet plate H2010 has a thickness H2010 of 5 μm and the liquid flow passage wall H1105 has a height H2011 of 5 μm.

Each of the lengths H2008 and H2009 with respect to the cut-away portion H1109 can be adjusted between, e.g., ((diameter H2005)×π/8)+1.5 μm (corresponding to the case of the cut-away portion vertex angle of 90 degrees) and ((diameter H2005)×π/8)+2.12 μm (corresponding to the case of the cut-away portion vertex angle of 60 degrees).

### (Liquid Ejecting Method)

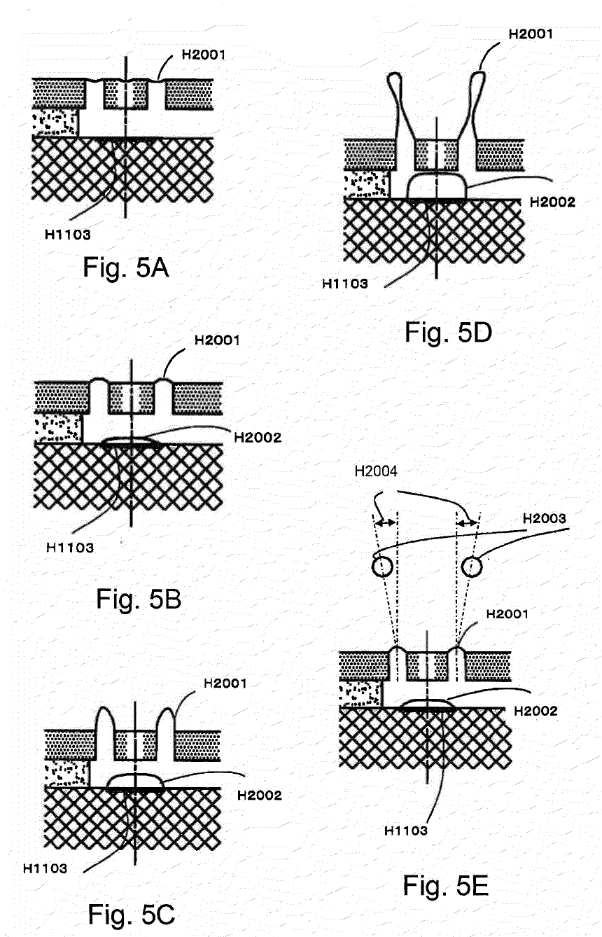

Next, an ejection operation of the liquid ejecting head having the above-described constitution will be described. FIGS. 5A to 5E are schematic views for illustrating a state of liquid ejection when the liquid ejecting head shown in FIGS. 4A to 4C is driven.

Before ejection, as shown in FIG. 5A, a meniscus H2001 is formed at each ejection outlet H1108 of the ejection outlet plate H1107. Then, when a voltage is applied to the heat generating element H 1103 in order to ejecting liquid, as shown in FIG. 5B, the heat generating element H1103 generates heat, so that liquid contacting the surface of the heat generating element H1103 in the liquid flow passage H1106 is heated to cause film boiling, thus generating a bubble H2002.

When the bubble H2002 is generated by the film boiling, a volume of the bubble abruptly grows, so that the liquid moves toward a downstream side (the ejection outlet H1108 side) and an upstream side (the liquid supply port side).

The ejection outlet H1108 is, as shown in FIG. 4C, asymmetrical by the presence of the cut-away portion H1109, thus being not cylindrical in shape. When a wall area ((the length H2007)×(the plate thickness H2010)) is taken as S1 and a wall area ((a total of the lengths H2008 and H2009)×(the plate thickness)) is taken as S2, S1<S2 is satisfied.

When the liquid passes through the ejection outlet H1108, due to a difference in wall area between the wall areas S1 and S2, a flow resistance difference is caused, so that a difference in flow speed of the liquid in the ejection outlet is caused. When the flow speed of the liquid flowing on the wall area S1 side is taken as V1 and that on the wall area S2 side is taken as V2, the liquid flow speeds V1 and V2 in the ejection outlet H1108 satisfy: V2<V1.

As the cut-away portion vertex angle H2014 is an acuter angle, the wall area S2 is increased, so that a flow speed difference (V1−V2) of the liquid in the ejection outlet H1108 is also increased.

As shown in FIG. 5D, a highest point of the meniscus H2001 is moved toward the cut-away portion H1109 side due to the flow speed difference between V1 and V2 of the liquid in the ejection outlet and then, as shown in FIG. 5E, a liquid droplet H2003 is traveled from a vertical direction of the heat generating element H1103 surface toward the highest point of the cut-away portion H1109. In the above constitution, by adjusting the cut-away portion, it is possible, to control a liquid droplet travelling angle H2004, e.g., in a range of 4 to 8 degrees as shown in FIG. 5E.

### (Ejection Outlet)

In First Embodiment, an ejecting direction of the liquid droplet is adjusted by providing the cut-away portion as described above but the cross-sectional configuration of the ejection outlet in the present invention is not limited to that described in this embodiment. As described above, the liquid droplet ejecting direction from the ejection outlet is deviated in angle from the vertical direction of the heat generating element surface. This may be attributable to such a cross-sectional configuration of the ejection outlet that it has no rotation symmetry, i.e., a rotationally asymmetrical cross section of the ejection outlet. That is, in order to vertically eject the liquid droplet with respect to the heat generating element, surface, the cross-sectional configuration of the ejection outlet is required to have rotation symmetry and to be well balanced. Conversely, the cross-sectional configuration of the ejection outlet is designed to have no rotation symmetry, thus disturbing the balance. As a result, it is possible to deviate the ejecting direction from the vertical direction of the heat generating element surface. Therefore, the ejecting direction can be controlled by adjusting the cross-sectional configuration of the ejection outlet, so that the cross-sectional configuration of the ejection outlet in the present invention can be adjusted so as to provide an intended eject direction of the liquid droplet.

As the cross-sectional configuration of the ejection outlet, as described above, the shape having no rotation symmetry can be employed. The cross-section of the ejection outlet may have e.g., a circular shape provided with a cut-away portion so that a distance from a center of the circular shape to a deepest point of the cut-away portion is longer than a radius of the circular shape.

The cut-away portion may, e.g., be defined by a plurality of rectilinear lines or a curved line connecting two points on a half-circle of the circular shape. The plurality of rectilinear lines or the curved line is located outside the circular shape.

For example, a cut-away portion constituted by two rectilinear lines corresponds to the cut-away portion employed in this embodiment. Specifically, the cut-away portion shown in FIG. 4C is constituted by two rectilinear lines each of which is a tangent line of the circle. These two rectilinear lines intersect at an angle of 90 degrees.

The shape of the ejection outlet may preferably be such a shape that the ejection outlet vertically passes through the ejection outlet plate with respect to the heat generating element surface in accordance with the cross-sectional configuration of the ejection outlet.

### Modified Embodiment 1

### Liquid Ejecting Head

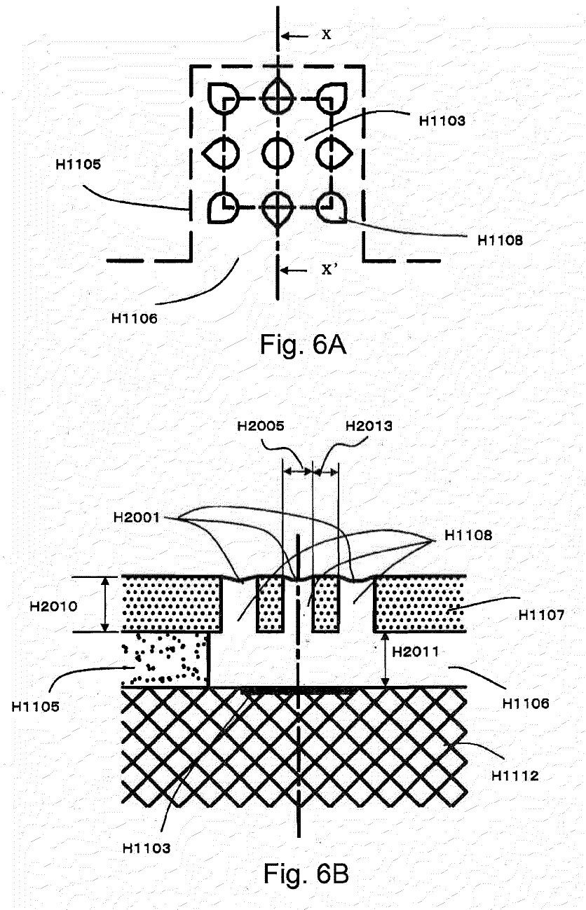

FIGS. 6A and 6B are schematic views for illustrating a modified embodiment of the liquid ejecting head shown in FIGS. 4A to 4C. In this embodiment shown in FIGS. 6A and 6B, a plurality of ejection outlets H1108 (nine ejection outlets in this embodiment) is provided with respect to one heat generating element. These ejection outlets are provided in such a manner that one ejection outlet is located at a center corresponding to a center of the heat generating element and eight ejection outlets are located on outermost effective ejecting power positions including four centers of four sides and four corners as shown in FIG. 6A. Each of the eight ejection outlets H1108 has a cut-away portion H1109 and is disposed so that a deepest point of each cut-away portion is most distant from the center of the ejection outlet. The remaining one (center) ejection outlet provided correspondingly to the center of the heat generating element has a circular shape.

Then, specific dimensions of the ejection outlets shown in FIGS. 6A and 6B will be described but the present invention is not limited thereto.

The heat generating element H1003 is a square having each side of 10 μm. Dimensions of the ejection outlet H1108 and the cut-away portion H1109 are the same as those described in Embodiment 1.

The ejection outlet plate H2010 has a thickness H2010 of 5 μm and the liquid flow passage wall H1105 has a height H2011 of 5 μm. An interval H2013 between adjacent ejection outlets H1008 is 3 μm.

### (Liquid Ejecting Method)

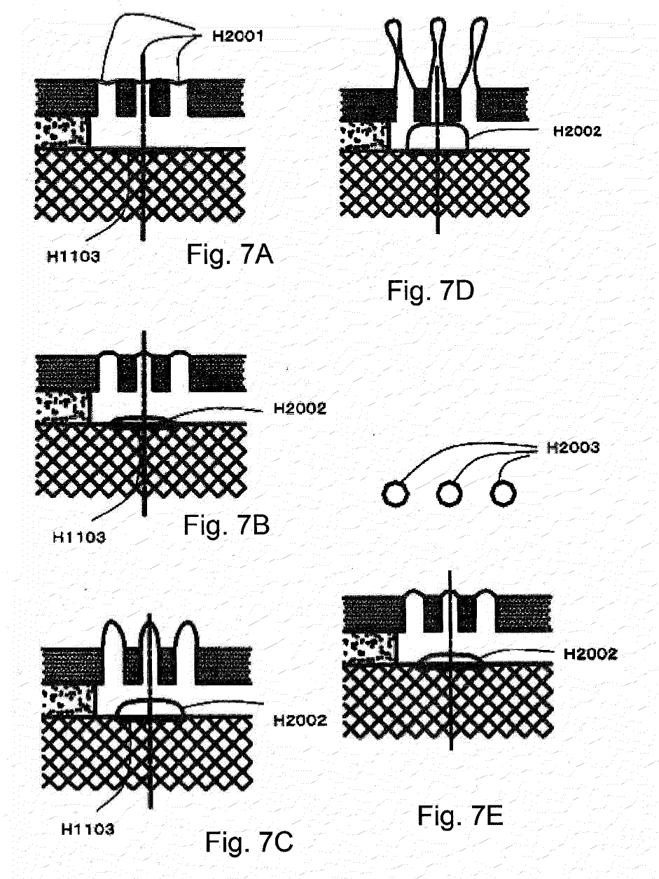

Next, an ejection operation of the liquid ejecting head having the above-described constitution will be described. The same constitution as that in Embodiment 1 is omitted from description. FIGS. 7A to 7E are schematic views for illustrating a state of liquid ejection when the liquid ejecting head shown in FIGS. 6A and 6B is driven.

As shown in FIG. 7D, a highest point of the meniscus H2001 is moved toward the cut-away portion H1109 side due to the flow speed difference (V1−V2) of the liquid in the ejection outlet and then, as shown in FIG. 7E, a liquid droplet H2003 is traveled from a vertical direction of the heat generating element H1103 surface toward the highest point of the cut-away portion H1109. In the above constitution, by adjusting the cut-away portion, it is possible to control a liquid droplet travelling angle H2004, e.g., in a range of 4 to 8 degrees as shown in FIG. 7E. The (true) circular ejection outlet H1108 having no cut-away portion H1109 permits ejection of a liquid droplet H2003 in a direction perpendicular to the heat generating element H1103 surface. The liquid droplets H2003 ejected from the plurality of ejection outlets H1008 can travel in arbitrary orbit.

By the above-described effects, in the case where the plurality of liquid droplets H2003 is caused to travel, the liquid droplets mutually travel in different directions even when the interval H2013 between adjacent ejection outlets is 5 μm or less, so that it is possible to provide a liquid ejecting head in which ejection outlets can be arranged in a narrow area at a high density without causing contact between the ejected liquid droplet.

### Modified Embodiment 2

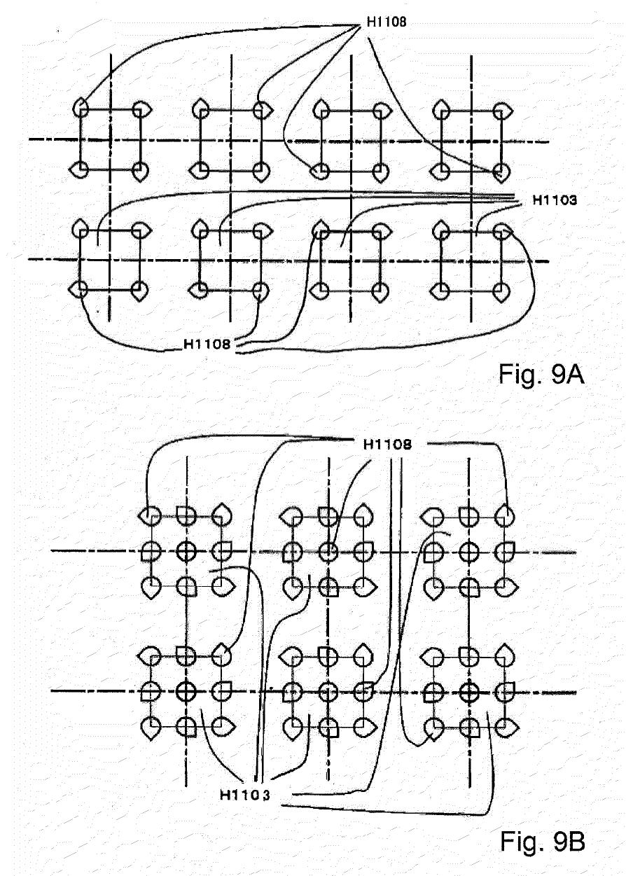

FIGS. 9A and 9B are schematic views each showing an arrangement example plurality of liquid ejecting heads in this embodiment.

FIG. 9A shows such an arrangement that ejection outlets H1108 each shown in FIG. 4C are disposed above four corners of square heat generating elements H1103 with the intention that deepest points of adjacent cut-away portions H1109 of ejection outlets H1108 are not close to each other. That is, in the case where a plurality of liquid ejecting heads each provided with four ejection outlets with respect to one heat generating element so that respective eject directions of liquid droplets do not contact each other, the ejecting directions of the ejection outlets are adjusted so that liquid droplet ejecting directions of ejection outlets of a liquid ejecting head and those of an adjacent liquid ejecting head do not contact each other.

As a result, the liquid droplets ejected from the ejection outlets of some liquid ejecting head travel without contacting those of an adjacent liquid ejecting head, so that a required liquid droplet diameter can be obtained. Therefore, it becomes possible to arrange the ejection outlets at a high density.

FIG. 9B shows such an arrangement that ejection outlets H1108 shown in FIG. 6A are disposed above a center, centers of four sides, and four corners of each of square heat generating elements H1103 with the intention that deepest points of adjacent cut-away portions H1109 of ejection outlets H1108 are not close to each other. Above the center of the square heat generating element H1103, a true circle ejection outlet is disposed. That is, in the case where a plurality of liquid ejecting heads each provided with nine ejection outlets with respect to one heat generating element so that respective eject directions of liquid droplets do not contact each other, the ejecting directions of the ejection outlets are adjusted so that liquid droplet ejecting directions of ejection outlets of a liquid ejecting head and those of an adjacent liquid ejecting head do not contact each other.

As a result, the liquid droplets ejected from the ejection outlets of some liquid ejecting head travel without contacting those of an adjacent liquid ejecting head, so that a required liquid droplet diameter can be obtained. Therefore, it becomes possible to arrange the ejection outlets at a high density.

### Embodiment 2

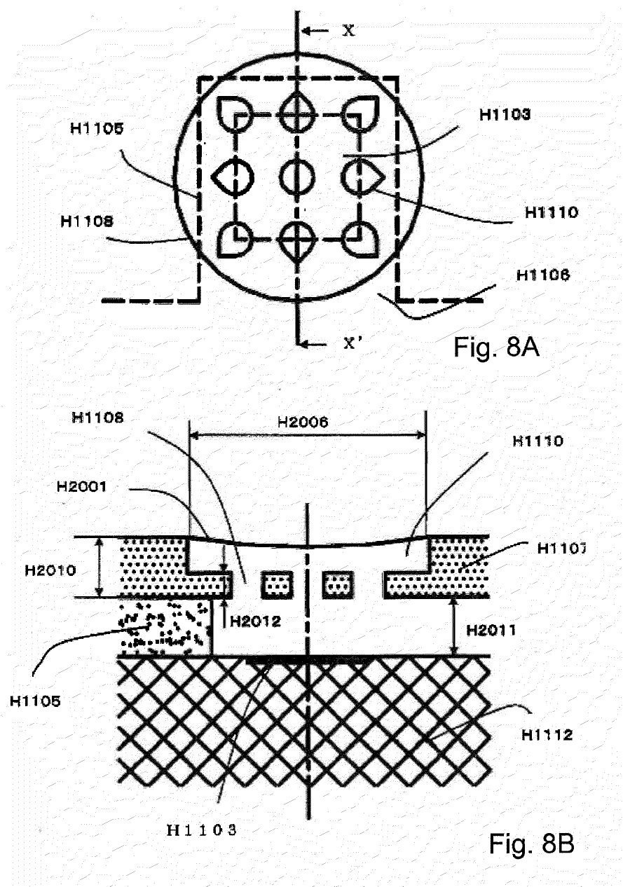

FIGS. 8A and 8B show a shape of a liquid ejecting head in this embodiment. In this embodiment, a recessed portion is provided on the ejection outlets, described in Embodiment 1 and Modified Embodiments 1 and 2, with a meniscus and the ejection outlets are located in liquid. That is, the ejection outlets are provided at a bottom of the recessed portion with the meniscus.

### (Structure of Liquid Ejecting Head)

Specific dimensions of the liquid ejecting head shown in FIGS. 8A and 8B will be described.

A heat generating element H1103 has a square shape having four sides each having a length of 10 μm and the recessed portion has a diameter H2006 of 18 μm. A shape and arrangement of the ejection outlets are the same as those in Modified Embodiment 1. The ejection outlet has a thickness H2012 of 1 μm. Further, an ejection outlet plate H1107 has a thickness of 5 μm and a liquid flow passage wall H1105 has a height H2011 of 5 μm.

It is preferable that the ejection outlet plate thickness H2010 and the liquid flow passage wall height H2011 satisfy: H2010≦H2011. In the case of the liquid ejecting head shown in FIGS. 8A and 8B, as described above, the ejection outlet plate thickness H2010 is 1 μm and the liquid flow passage wall height H2011 is 5 μm. Therefore, it is understood that the relationship: H2010≦H2011 is satisfied.

### (Ejecting Method)

In the above-described liquid ejecting head, before ejection, a meniscus H2001 is formed on the recessed portion H1110 and the ejection outlets H1008 are located in the liquid as described above. Then, when the heat generating element H1103 is driven for liquid ejection, the liquid is moved toward at least the ejection outlet side.

During the movement of the liquid toward the ejection outlet side, the liquid passes through the ejection outlet H1008. As a result, a liquid moving speed after the passing of the liquid through the ejection outlet is markedly higher than that before the passage of the liquid through the ejection outlet, so that a flow speed of the liquid in the recessed portion is relatively increased.

Therefore, the meniscus of the recessed portion partly rises at portions opposing the ejection outlets, so that liquid droplets are ejected. In this case, the ejection outlet H1008 is provided with the cut-away portion H1009, so that orbit of the liquid droplet even in the case of ejection of the liquid droplet from the recessed portion is such that the liquid droplet travels toward the deepest point of the cut-away portion H1009.

That is, the entire ejection of the liquid in the recessed portion is not caused to occur, so that it is possible to eject a plurality of minute liquid droplets (e.g., 0.014 pico-liter (pl)). Further, the plurality of liquid droplets travels in different directions, so that the liquid droplets do not contact each other and therefore it is possible to obtain liquid droplets each having an intended size. Thus, it is possible to provide liquid ejecting heads which can be arranged in a narrow area at a high density.

Further, the liquid is retained in the recessed portion so that the ejection outlets H1108 are located in the liquid. For this reason, the ejection outlets do not cause clogging by drying of the liquid, so that good liquid droplets can be ejected from start of the ejection.

### INDUSTRIAL APPLICABILITY

According to the present invention, even in the case where the ejection outlets are arranged at a high density, it is possible to prevent contact between liquid droplets by changing liquid droplet ejecting directions of the respective ejection outlets. As a result, it is possible to provide a liquid ejecting head device capable of ejecting minute liquid droplets at a higher density than the case of the conventional liquid ejecting head devices.

While the invention has been described with reference to the structures disclosed herein, it is not confined to the details set forth and this application is intended to cover such modifications or changes as may come within the purpose of the improvements or the scope of the following claims.

1. A liquid ejecting head comprising:
an energy generating element for generating energy for ejecting liquid containing a medicine; and
a plurality of ejection outlets, provided for said energy generating element, for ejecting the liquid,
wherein each of said ejection outlets has a rotationally asymmetrical cross-section in a plane parallel to said energy generating element.

2. A head according to claim 1, wherein the cross-section has a shape such that a circular shape provided with a cut-away portion so that a distance from a center of the circular shape to a deepest point of the cut-away portion is longer than a radius of the circular shape.

3. A head according to claim 2, wherein the cut-away portion is defined by a plurality of rectilinear lines or a curved line connecting two points on a half-circle of the circular shape.

4. A head according to claim 1, wherein said energy generating element is a heat generating element or a vibrational energy generating element.

5. A head according to claim 1, wherein each ejection outlet is provided at a bottom of a recessed portion with a meniscus and is located in the liquid.

6. A head according to claim 2, wherein said ejection outlets provided for said energy generating element are different in direction from the center of the circular shape to the deepest point of the cut-away portion.

7. A head according to claim 1, wherein said ejection outlets provided for said energy generating element includes at least one ejection outlet having a circular cross section.
